(19) United States
(12) Patent Application Publication
(10) Pub. No.: US 2016/0101194 A1
(43) Pub. Date: Apr. 14, 2016

(54) SMART® Medication Adherence Formulation, Method, Device and System for Topical, Vaginal or Rectal Routes of Administration

(71) Applicant: University of Florida Research Foundation, Incorporated, Gainesville, FL (US)

(72) Inventor: Donn M. Dennis, Gainesville, FL (US)

(73) Assignee: University of Florida Research Foundation, Incorporated, Gainesville, FL (US)

(21) Appl. No.: 14/785,888

(22) PCT Filed: May 21, 2014
(86) PCT No.: PCT/US14/39001

§ 371 Date: Oct. 21, 2015

Related U.S. Application Data
(60) Provisional application No. 61/828,975, filed on May 30, 2013.

(51) Int. Cl. A61K 49/00; G01N 33/497

(52) U.S. Cl. CPC A61K 49/00; G01N 33/497; G01N 2033/4975

(57) ABSTRACT

Novel compositions, methods, systems and devices are disclosed which contain markers for definitive medication adherence monitoring for Active Pharmaceutical Ingredients (APIs) delivered topically, vaginally or rectally. This invention is useful in a wide range of contexts, including, but not limited to, clinical trial settings, home use settings, or other settings, where it is necessary to definitively confirm that a given patient has taken or been administered a given medication at the correct time and in the correct dosage via a topical, vaginal or rectal route of delivery. Specific formulations of markers are disclosed for inclusion in compositions for Active Pharmaceutical Ingredient (API) delivery, including but not limited to delivery of microbicidally active compounds such that on topical, vaginal or rectal delivery, said AEM is detected in the breath or an Exhaled Drug Emplacement Marker, EDEM, which is a metabolite of the AEM, is detected in the breath.

### FIELD OF THE INVENTION

Methods, systems, devices and formulations to facilitate definitive documentation of medication adherence for products administered by vaginal or rectal routes.

### BACKGROUND OF THE INVENTION

In HIV pre-exposure prophylaxis (PrEP) trials such as VOICE, sub-optimal oral and vaginal product adherence has precluded accurate estimation of drug efficacy. (van der Straten A, Van Damme L, Haberer J E, Bangsberg D R. Unraveling the divergent results of pre-exposure prophylaxis trials for HIV prevention. AIDS. 2012 April 24; 26(7):F13-9). Measurement of adherence is a major, unmet challenge. Other objective measures of adherence in microbicide trials have been tested, but none measure the actual use of the product. This invention provides a breath-based, adherence monitoring system and composition for investigators studying vaginal and rectal routes of drug (e.g., microbicide) administration and for deployment in the field to ensure such routes of delivery are adhered to.

In WO2013/040494, published 21 Mar. 2013, entitled “SMART™ SOLID ORAL DOSAGE FORMS”, a number of physical forms for delivery of active therapeutic agents in combination with markers were disclosed. As disclosed in that publication, SMART® stands for Self Monitoring And Reporting Therapeutics. The SMART® system involves use of proprietary breath gas analyzers for medical diagnostics for verifying ingestion of medications using patient biometric identification, including detection in the exhaled breath of compounds included in or produced from the ingested medication.

The SMART® adherence system accurately confirms whether the right person took the right dose of the right drug via the right route at the right time. We call this type of adherence assessment “definitive” because it would be very difficult, if not impossible, for subjects to deceive the system. The SMART® system reliably indicates that the correct person actually self administered the drug or was administered the medication, for instance, by a caregiver.

The SMART® system, essentially a personalized medicine tool that provides a significantly better understanding of drug safety and efficacy, is designed to operate in all clinical trial and disease management environments, including the home. It contains two key components: 1) the SMART® drug, which includes or generates a marker or markers that appears in the exhaled breath of humans or other vertebrates, termed herein Exhaled Drug Ingestion Markers (EDIMs) to confirm definitive medication adherence, and 2) the SMART® device, which accurately measures the EDIMs, provides medication reminder functions, and orchestrates critical adherence information flow between the relevant stakeholders.

Typical sensing technologies used to measure EDIMs include but are not limited to miniaturized Gas Chromatography linked to a Metal Oxide Sensor (mGC-MOS), surface acoustic wave (SAW) sensors, ion mobility spectroscopy (IMS) sensors, infrared (IR) sensors and the like. Elements of this art have been broadly taught in previous patent applications and issued patents: See, for example, “Marker detection method and apparatus to monitor drug compliance”, U.S. Pat. No. 7,820,108; US 2005/0233459; “System and Method of Monitoring Health Using Exhaled Breath”, US2007016785; “Methods and Systems for Preventing Diversion of Prescription Drugs”, US20080059226; and “Medication Adherence Monitoring System”, US 2010/0255598.

In order for pharmaceutical companies and the general public to be able to broadly utilize the SMART® system for adherence using rectal or vaginal routes of drug delivery, in clinical trials, and/or in disease management or prevention, novel strategies to package Adherence Enabling Markers (AEMs, preferably Generally Regarded As Safe, GRAS compounds) with clinical trial materials (CTMs) and marketed drugs should meet at least the following two criteria:

1) The method of packaging specific GRAS compounds (taggants) as AEMs with the medication should provide a tamper resistant (literally foolproof) measurement of adherence that is highly accurate; and
2) The method of packaging the AEMs should ideally not alter the manufacturing processes of the CTM or marketed drug, and require minimal-to-no changes in their chemical, manufacturing, and controls (CMC).

In recent years, it has become increasingly clear that adherence to microbicide gel use is critical to optimizing effectiveness in preventing human immunodeficiency virus (HIV) transmission. HIV and AIDS remain an important cause of morbidity and mortality around the world. Recently, a proof-of-concept trial (CAPRISA 004) demonstrated that pericoital use of 1% tenofovir gel was associated with 54%, 38%, and 28% reductions in HIV acquisition in women with high (>80%), intermediate (50%-80%), and low (<50%) adherence, respectively, compared with placebo. (Abdool et al., “Effectiveness and safety of tenofovir gel, an antiretroviral microbicide, for the prevention of HIV infection in women.” Science, (2010)329 (5996):1168-1168). In that investigation, adherence was measured by self-report and by assessing subjects' return of used and unused gel applicators. Indeed, preexposure adherence to antiretroviral agents has important implications for clinical studies, as poor adherence is one of the primary sources of “efficacy dilution” in clinical trials of vaginal microbicides, obscures safety data as subjects with adverse effects may stop using the agent, and prevents future improvements in clinical trial design to address issues related to adherence. (Pool et al. “Assessing the accuracy of adherence and sexual behavior data in the MDP301 vaginal microbicides trial using a mixed methods and triangulation model.” PLoS One. 2010; 5(7):e11632; Masse et al, “Efficacy dilution in randomized placebo-controlled vaginal microbicide trials.” Emerg Themes Epidemiol. 2009; 6:5). Moreover, Harter and Peck (Harter J G, Peck C C. “Chronobiology: suggestions for integrating it into drug development.” Ann N Y Acad Sci. 1991; 618:563-571) used error propagation theory applied to clinical trials to demonstrate that subject adherence is the single greatest contributor to biological variation in studies because non-adherence propagates into larger errors in pharmacokinetic and pharmacodynamic analysis. For these reasons, adherence endures as a paramount issue, not just in disease management but also in clinical trial design of HIV prevention interventions and other therapeutic modalities.

Breath represents an almost ideal diagnostic matrix with several favorable properties, including easy access across all populations, availability of large volumes of specimens, cleanliness, ability to collect in non-private locations, relatively simple techniques to collect, ease of handling, low power transfer/measurement devices, and others. One strategy to address adherence using a breath matrix has been hypothesized to be addition of a taggant (AEM) to the vaginal or rectal gel. The presence of the AEM or its metabolic product exhaled in breath that can be sensitively and specifically detected by portable sensors would document use of the vaginal or rectal gel. The presence of the AEM or metabolite in breath indicates the release of the AEM from the vaginal or rectal product, and absorption of the AEM across the vaginal or rectal mucosal barriers into the vascular compartment (blood). Depending on the taggant used and its dose, the duration of the presence of the volatile marker in the breath may be varied according to need. In addition, the possibility of taggants in oral intake (e.g., food, beverages) may affect selection of an appropriate taggant.

In Morey et al., J. Clin. Pharmacol., (2013), V. 53, no. 1, pp. 103-111, “Feasibility of a Breath Test for Monitoring Adherence to Vaginal Administration of Antiretroviral Microbicide Gels”, and in van der Straten, (2013), AIDS Behav. (Epub ahead of print), “A Novel Breath Test to Directly Measure Use of Vaginal Gel and Condoms”, ester taggants (2-butyl acetate, 2-pentyl acetate, isopropyl butyrate, and 2-pentyl butyrate) added to vaginal gels were tested for generation of exhaled secondary alcohol and ketone metabolites to potentially provide a “breath test” for vaginal gel use. With some variations in these two studies (intravaginal taggant delivery by gel alone or use of a condom, and testing of whether dermal administration might confound the system), breath samples were collected using bags before and after taggant administration with the vaginal gel. Samples were measured using a miniature gas chromatograph and/or gas chromatography-mass spectroscopy for ester taggant, alcohol, and ketone concentrations. After vaginal administration, the parent ester and metabolites for the acetate esters were observed in breath, whereas isopropyl butyrate, 2-pentyl butyrate, and metabolites were not. In addition, some women reported self-resolving, mild burning with vaginal administration or a “bubblegum” taste. No taggants or metabolites were detected in breath following dermal application. It was concluded in each of these studies that a “breath test” for adherence to antiretroviral vaginal gel application may be physiologically and technically feasible. However, the adverse reception of the taggants by the test subjects was problematic. In addition, these studies demonstrated the inability to measure ester taggants on the breath following dermal application.

To make this test useful in the clinical trials, a simple method to measure taggants and analytes must be available, and the addition of the taggant has to be well-received by subjects, and the gel or other medium itself and any included Active Pharmaceutical Ingredient (API) has to be unaffected by the addition of the taggant.

An improved composition, method and system of assessing medication adherence, consisting of tagging a medication gel, transdermal composition, rectal administration composition, vaginal administration composition, or the like, collecting the taggant or metabolite(s) in breath, and measuring that taggant or metabolite(s) in breath, is disclosed and claimed herein. Thus, this technology provides clinical trial investigators and health care practitioners with high-quality data about subjects'—or patients'—definitive adherence. The technology described herein, which represents a refinement and novel composition for achieving the goals at the heart of the SMART® system by providing a novel formulation, composition of matter and methods, over and above that which is disclosed in the art outlined herein above, for adherence monitoring when non-oral routes of medication administration are appropriate, provides an invention to accomplish both of these goals.

### SUMMARY OF THE INVENTION

This patent disclosure provides detailed disclosure for production of novel dosage forms for non-oral administration of medications which contain markers for definitive medication adherence monitoring. The novel Non-Oral Dosage Forms (NODFs) are useful in a wide range of contexts, including, but not limited to, clinical trial settings, home use settings, or other settings, where it is necessary to definitively confirm that a given patient has taken or been administered a microbicidal medication (or other high-value pharmaceutical, including but not limited to small molecules, peptides, proteins, (natural, synthetic or recombinantly produced), DNA-based, RNA-based therapeutic agents, such as aptamers, for use as Active Pharmaceutical Ingredients (APIs)) at the correct time and in the correct dosage.

The present invention provides specific formulations of markers for inclusion in a variety of non-oral dosage forms which ensure rapid or timed-release delivery of relevant Active Pharmaceutical Ingredients (APIs) in association with an Adherence Enabling Marker, “AEM”. Exemplary embodiments are disclosed herein.

Accordingly, it is an object of this invention to provide novel Non-Oral Dosage Forms (NODFs) comprising chemistries that optimize the efficacy of SMART® (Self Monitoring and Reporting Therapeutics) systems.

Another object of this invention is to provide novel combinations of SMART® markers.

Another object of this invention is to provide compositions, systems and methodology for application of SMART® technology to medication adherence monitoring, while requiring minimal modification of the regulatory profile for Active Pharmaceutical Ingredients (APIs) when delivered in non-oral dosage forms, including via vaginal, rectal, or other non-oral routes of delivery.

Other objects and advantages of this invention will be apparent to those of skill in the art from a review of the entire disclosure and the appended claims.

### BRIEF DESCRIPTION OF THE DRAWINGS

FIG. 1. Provides an illustrative example of how the system and method according to this invention works when medication containing an Adherence Enabling Marker is delivered orally. While this figure is taken from a published document, the Adherence Enabling Marker (AEM) composition according to this invention was not available in the art at the time that the publication from which this figure is taken was generated. The figure illustrates enzymatic catalysis and resultant exhalation of 2-butanone following oral ingestion of 2-butanol (40 mg) in subjects (n=7). Panel A: metabolism of the AEM, 2-butanol, by αα-alcohol dehydrogenase (ADH) to generate the volatile product, 2-butanone, an Exhaled Drug Ingestion Marker (EDIM). Panel B: breath concentration-time relationship for the exhalation of 2-butanone (an EDIM) in breath following consumption of 2-butanol at time 0 min. Data shown are mean±SD. *, P<0.05 for a given time point compared to time point 0 min. The arrow denotes time of capsule ingestion. Concentrations less than the level of 1.0 parts-per-billion (ppb) are noted as “<LOD”. The present invention provides a method for achieving adherence monitoring when medication is delivered by non-oral routes of delivery, including via vaginal and/or rectal routes.

FIG. 2. A graphic showing various devices for separation and co-administration of an AEM and an API via the vagina or rectum such that an EDEM is detected on the exhaled breath.

FIG. 3. Breath kinetics of exhaled d6-acetone following topical application of d8-isopropanol (d8-IPA) in a carbomer gel or oral ingestion of d8-isopropanol; left axis=100 mg d8-IPA oral; right axis, 20 mg d8-IPA oral and 240 mg d8-IPA topical.

FIG. 4. Exhaled acetone and 2-butanone following rectal administration of 40 mg of 2-butanol in 3 mL HEC (hydroxyethylcellulose) gel.

### DETAILED DISCLOSURE OF THE PREFERRED EMBODIMENTS OF THE INVENTION

Xhale, Inc., (http://www.xhale.com) has developed a breath-based technology, the SMART® (Self Monitoring and Reporting Therapeutics) Adherence System, to monitor individual subject, dose-by-dose, oral medication adherence in real-time. The SMART® system uses FDA-approved additives, termed adherence-enabling markers (AEMs), which generate volatile metabolites in vivo that are exhaled by a subject. Measurement of these metabolites in a breath sample unambiguously documents ingestion of oral drugs. The AEMs, FDA designated Generally Recognized as Safe (GRAS) compounds, are formulated with APIs in a manner that alters neither the drug's manufacturing processes nor bioavailability by whatever form of delivery, whether that be rectally, via, for example a suppository formulation; vaginally, as in a vaginally applied gel, condom coating or the like; or transdermally, as in, for example, via a transdermal patch.

A. SMART® and Oral Routes of AEM Administration

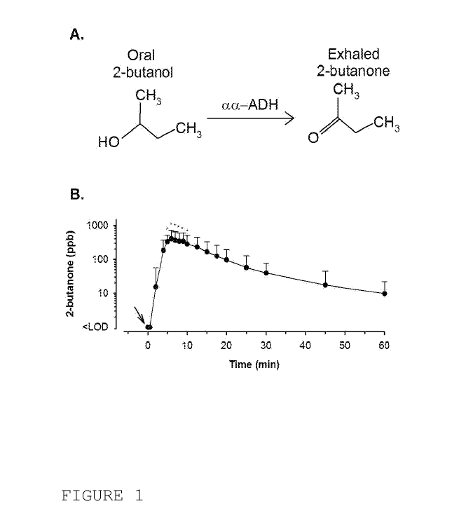

Once orally administered, the AEM(s) is (are) absorbed at the site of delivery and is (are) metabolized to a volatile marker(s) that is rapidly exhaled in breath (see FIG. 1). The concentration(s) of the metabolite(s) in the breath sample (˜20 mL) is then automatically measured by a portable, lightweight, miniature gas chromatograph (mGC)—the SMART® device—without subject effort. By measuring the metabolite(s) in breath, one can be assured that the subject did, indeed, consume the medication because native gastric wall and hepatic enzymes (e.g., αα-alcohol dehydrogenase) are needed to metabolize the AEM(s) to the volatile, exhaled metabolite(s), referred to herein as the Exhaled Drug Ingestion Marker, or EDIM. All data (date/time stamps, breath chromatographs, yes/no adherence assessments, mGC self-diagnostic quality assurance logs) are stored locally in the mGC device on an internal USB flash drive for later collection and/or transmitted in real-time using encrypted Health Insurance Portability and Accountability Act (HIPAA)-compliant wireless or cellular router technology to a central data repository for analysis. Two additional optional data streams are available to investigators should the study requirements warrant collection when compared to subject privacy concerns: 1) a camera in the SMART® device is time-gated to concurrent breath collection; this biometric authentication (facial picture) allows investigators to definitively confirm that the breath analyzed by the SMART® device originated from a specific subject, and, 2) the concentration of ethanol in a subject's breath sample that particularly interests investigators studying psychotropic drugs (developed under NIAAA 5R44AA017009). These data can likewise be stored locally on the SMART® device and/or transmitted to a data repository. Data are logged into custom-written, internet-based, HIPAA-compliant templates for review by authorized investigators anywhere on the globe with an internet connection. Investigators may choose to actively review the data on a daily basis to understand day-to-day adherence (active management), to maintain data securely in a blinded fashion until assignment unmasking (passive management), or some combination of active/passive review desired by the study team.

These data allow reasearchers to know if subjects were actually administering and using the assigned research article, and/or following scheduled dosing. This information is important when assessing the safety and efficacy of a drug. As a result dose-to-dose intervals and pharmacokinetic/pharmacometric drug modeling are available from this system to inform ongoing treatment modalities. The long-term health effects of suboptimal adherence to a drug could be assessed, motivations for adherence in different states (e.g., healthy/ill; home/travelling) could be investigated since adherence data by time/date is available for the first time. In addition, this system enables reliable study of the effects of behavioral interventions to improve adherence. Clinical investigators will likely discover other new uses for this system as it becomes available for full use in a broad swath of studies across multiple populations and locations. The key to understanding adherence, like any scientific data, is measuring it. The breath-based technology system provides this tool to scientists and clinical trial investigators.

From a participant's perspective, the adherence measurement system is easily portable and designed to be self-administered by subjects in their own residences or workplaces. This feature offers significant subject convenience and investigator economic benefits compared to frequent appointments with study staff for directly observed therapy (DOT), the “gold standard” of adherence. Additionally, since no study staff is required for daily assessments, this adherence system can be used at any time whereas DOT is generally available only during business hours and not during weekends or holidays. Overall, the change in subject behavior is simply a 5 sec breath exhalation into the mGC ≈10 min or less after administering the API-AEM composition. By altering AEM dose and/or type, the duration of marker persistence in breath can be adjusted, to maximize versatility of the SMART® system. All breath analyses and data logging/transmission are seamless to the subject and occurs automatically. Usability studies conducted under NIMH 2R44MH081767-02A1 indicated a high degree of satisfaction with this system by HIV/AIDS patients receiving adherence measurement for highly active antiretroviral therapy (HAART).

To date, Xhale, Inc. has focused its development efforts on commercial development of the SMART® adherence system for SODFs, particularly tablet- or capsule-based medications, which are swallowed, enter the stomach, and are absorbed in the gastrointestinal tract (see WO2013/040494). In this case, definitive adherence is indicated by the detection of a metabolite of an AEM, also referred to herein as a taggant (preferably a GRAS compound) which may also be the EDIM or which is the source for the production of the EDIM. The taggant is packaged together with the final SODF. In that embodiment, the SMART® system has successfully employed 1) various formulation strategies that incorporate taggants into the final dosage form without altering the manufacturing processes of the CTM or marketed drug per se and causing minimal-to-no change in their CMCs, and 2) a mGC-MOS as the SMART® device to measure the EDIMs.

Prior to describing the current invention, a brief review of some key aspects of taggant chemistry outlined in the above referenced patents is provided.

Consider a scenario where a patient with a specific disease ingests an active drug, A, for treatment, which is metabolized by enzyme(s) to Al plus other irrelevant metabolites. In this example, a safe taggant (e.g., GRAS flavorant) without pharmacological activity called T, which may be metabolized to a major metabolite, Tl plus other irrelevant metabolites, is packaged with A. Thus, the two relevant metabolic reactions are: 1: A→Al+others 2: T→Tl+others

With regard to measuring a marker (s) that appears in breath, the EDIM(s), which can be measured to verify that A was orally ingested by the patient, we have 4 obvious candidates: 1) A; 2) a major metabolite of A, Al; 3) a taggant, T, which was ingested with the medication containing A; or 4) a metabolite of any taggant (T), Tl, which was generated via enzyme metabolism of a taggant (T). The appearance of Tl about 5-10 min later in the breath can be used to document the active drug A (the Active Pharmaceutical Ingredient, or API) was actually ingested. To optimize performance of the adherence system, we have developed a novel composition of matter herein wherein a taggant is stably included in a soft gel capsule which is well tolerated by test subjects, which generates markers in the exhaled breath which are quickly and reliably detected, and which do not interfere with co-delivered APIs.

B. SMART® and Vaginal/Rectal Routes of API and AEM Administration

In developing the present invention, commercial imperatives relevant to manufacture and delivery of APIs via a vaginal and rectal route containing volatile marker molecules have been carefully considered, experimented with and optimized to achieve excellent methods for making and including AEM formulations, and deployment with APIs.

As noted in the background section of this disclosure with respect to attempts to deliver ester AEMs via dermal or vaginal routes, simple esters that were “acetate-based” as opposed to “butyrate-based” readily appeared in breath in a concentration and within a time frame that would be needed (and practical) for an adherence application for detecting microbicidal (vaginal or rectal) product placement (e.g., microbicide gel to prevent HIV transmission). The known ester-based AEMs, however, exhibited stinging and adverse taste experiences for subjects when the AEMs were delivered vaginally, and were very difficult to solubilize in the vaginal microbicide gel (see below for additional discussion). Thus, there is significant unpredictability in being able to successfully deliver AEMs via vaginal or rectal routes.

As a starting point for the present invention, the inventors hypothesized that the butyrate esters may have a greater barrier to permeability across the vaginal epithelium, whereas the acetate esters may more easily traverse this barrier to diffusion. For microbicide applications, gels in commercial use typically have high glycerin contents. While glycerin is a good medium for solubilization of alcohols, it is not a good solvent for esters. Unless one does the experiment, independent of the rectal/vaginal permeability issues, it was not known if the gel medium itself would sequester alcohols to such a degree that even if alcohol-based AEMs were tested, rather than ester-based AEMs, while potentially able to cross the vaginal or rectal lining barriers, may be released from the vaginal gel or other commercial formulation medium at such a slow rate of egress that they would not be amenable to adherence applications. The esters are difficult to solubilize in the gels, and frequently caused “stinging” upon vaginal application and tastes (food additives—bubble gum taste, etc) that were poorly received by the test subject. The secondary alcohols should not have these problems. In light of these elements of unpredictability in this art, it is surprising that the taggants and formulations disclosed and claimed herein work unexpectedly well for various microbicidal delivery.

Thus, while use of 2-butanol as a marker for SMART® system adherence was disclosed in WO2013/040494 for oral routes of delivery, the invention disclosed herein provides advancements in the art by resolving such matters as flashpoint and volatility of AEMs during formulation of the taggant, stability of incorporating the taggant into the formulation, acceptability of the AEM to subjects receiving administered medication, and confirms that non-toxic, preferably GRAS (Generally Recognized as Safe) secondary and tertiary alcohols with between three and up to eight carbon atoms are excellent AEMs for non-oral routes of AEM delivery, including but not limited to vaginal and rectal routes. In a preferred embodiment, the GRAS compound is, ideally, a direct food additive.

C. The SMART® AEM Compositions According to the Present Invention, Methods of Manufacture and Use Thereof with the SMART® Adherence System to Definitively Document Adherence for Vaginally and Rectally Delivered APIs

In this disclosure, what is detected on the exhaled breath of a subject following vaginal or rectal delivery of a composition or device according to this invention is termed an Exhaled Drug Emplacement Marker, or EDEM, rather than being referred to as an Exhaled Drug Ingestion Marker, or EDIM. This is purely a semantic difference in order to more accurately describe the origin of the marker, since, with vaginal or rectal delivery, ingestion may not be considered an appropriate descriptor. For purposes of operation of the SMART® system, however, the terms EDIM and EDEM should be considered interchangeable—in either case, what is intended is a volatile compound which is detected in the exhaled breath of a subject following administration of a composition comprising an Adherence Enabling Marker, or AEM, which itself may be the EDIM/EDEM or which is metabolized to produce the EDIM/EDEM. Accordingly, for all intents and purposes, these terms are interchangeable and are used differentially based on the context and site of drug/AEM delivery/ingestion.

Within this disclosure, while considerable written description and attention is focused around use of 2-butanol as an Adherence Enabling Marker (AEM) for generation of Exhaled Drug Emplacement Markers (EDEMs) (which, in the case of 2-butanol as the AEM is 2-butanol itself and the ketone, 2-butanone; due to much less 1^{st} pass metabolism than when delivered via an oral route, different patterns of 2-butanol to 2-butanone in blood and hence breath arise), which is/are detected in the exhaled breath following vaginal or rectal application of medication formulated with the AEM, those skilled in the art will appreciate that other AEMs and EDEMs may be similarly used for this purpose.

Generally, non-toxic, and preferably GRAS secondary and tertiary alcohols with between three and up to eight carbon atoms are useful for this purpose. Thus, for example, any or each of the following compounds may be used according to this invention as an AEM for non-oral delivery of AEMs for use in combination with the SMART® system: isopropanol; 2-butanol; 2-methyl-2-butanol; 2-pentanol; 3-pentanol, etc. Preferred secondary and tertiary alcohols are those that are GRAS compounds, and any of those compounds listed below in Table I may be selected for this purpose.

In addition, while the present disclosure focuses on specific excipients and combinations thereof with the AEMs disclosed herein, those skilled in the art will appreciate that other equivalent excipients may be utilized with the disclosed AEMs.

An optimized AEM composition is disclosed herein which comprises at least or exclusively the following key components, mixed either prior to delivery or at the site of delivery at an appropriate concentration with a vaginal or rectal gel or other appropriate medium known in the art or which hereafter comes to be known in the art:

a. An AEM, primarily exemplified herein by 2-butanol, but which may be any of the AEMs listed in Table I;
b. A gel medium for delivery of the AEM and/or Active Pharmaceutical Ingredient (API);
c. At least one API, unless the AEM is being delivered in a placebo.

As noted above, those skilled in the art will appreciate that AEMs other than 2-butanol, including those shown in Table I below, may be appropriate for a particular application and can, based on the disclosure and guidance provided herein, make appropriate modifications to the formulation to accommodate alternate AEMs, volumes, concentrations and chemical interactions. When delivering the AEM via a vaginal or rectal route, particularly where an anti-HIV API is being co-delivered with the AEM, it is critical to ensure that the amount and concentration of secondary or tertiary alcohol acting as the AEM be so low as to avoid inflammatory responses known to be caused when high concentrations and amounts of alcohol, e.g. ethanol, is delivered via these routes. This is because it is known that high concentrations of alcohol when introduced into the vagina or rectum, while able to cross the cellular barrier, induce significant inflammation. Aside from the associated discomfort, this also reduces a critical natural barrier to infection—actually increasing the susceptibility to infection by, for example, HIV.

Surprisingly, successful detection of EDEMs in exhaled breath is achieved following inclusion of as little as about 3 to 10 mg of 2-butanol. These doses, especially when dissolved in standard volumes of microbicide gel (typically 4 ml), are very unlikely to elicit any inflammatory response at the site of delivery. For example, when a dose range of about 3 to 30 mg of 2-butanol is delivered vaginally or rectally in an appropriate carrier medium, e.g., tenofovir placebo gel (i.e. the same medium in which tenofovir is delivered but with or without the active agent tenofovir) even more reliable detection of 2-butanol and 2-butanone in the exhaled breath is achieved in a time frame and concentration sufficient to definitively confirm product placement with a high level of confidence, and without induction of inflammation at the delivery site. While greater amounts of AEM could be delivered by this route without causing inflammation, it is preferred to delivery no more than 100 mg of AEM, and, most preferably, to deliver between about 3 to 30 mg, and, most preferably, to deliver between about 3 and 30 mg.

Since the physiology of the vaginal lining includes a significant barrier to delivery and diffusion of AEMs and APIs, due to the thick, stratified squamous epithelial lining, and yet the inventors herein are able to successfully deliver AEMs via this route, rectal delivery, where a single epithelial cell layer forms the surface of the rectum, is assured. Compositions, means and devices for rectal delivery include gels, as for vaginal delivery, and such dosage forms as suppositories, which may include the API in an appropriate suppository vehicle known in the art, with the AEM admixed therein or in a separate suppository compartment, coating or the like.

In formulating the AEM according to this invention for vaginal or rectal delivery concurrently with an API, it is important to utilize gels, lubricants, vehicles, and the like for AEM/API delivery which do not enhance transmission of disease causing agents, such as HIV. For example, see Begay et al., “Identification of Personal Lubricants That Can Cause Rectal Epithelial Cell Damage and Enhance HIV Type 1 Replication in Vitro”, AIDS Research and Human Retroviruses, Volume: 27 Issue 9: Aug. 23, 2011, which found that many over-the-counter personal lubricants damage epithelial linings and, in some cases, enhance HIV-1 replication. The same or similar formulation as used for Tenofovir placebo gel may be used with substitution of a small fraction of the glycerol with the preferred alcohol according to this invention. From a chemical standpoint the alcohol substitutes very well for glycerol in these systems, and ensures excellent compatibility and solubility of even higher doses of alcohols.

Different AEM's may be included in a single composition in order to permit differential kinetics of appearance in breath to be optimized. Thus, more complex AEMs (higher carbon atom content) generally exhibit longer half life in the breath, whereas the smaller, simpler AEM's are more quickly cleared from the breath. Understanding these kinetic considerations will permit those skilled in the art, based on the present disclosure, to select different AEMs and combinations of AEMs, in order to tailor detection kinetics in the breath for monitoring adherence with respect particular APIs and different modes of clinical use. In addition, or alternatively, a mixture of different APIs in a delivery medium or substrate, wherein each API is associated with a different AEM, may be utilized, and thereby, delivery of each API may be tracked by detection of distinct markers on the breath, even if/when a mixture is prepared for delivery of several different APIs/AEMs.

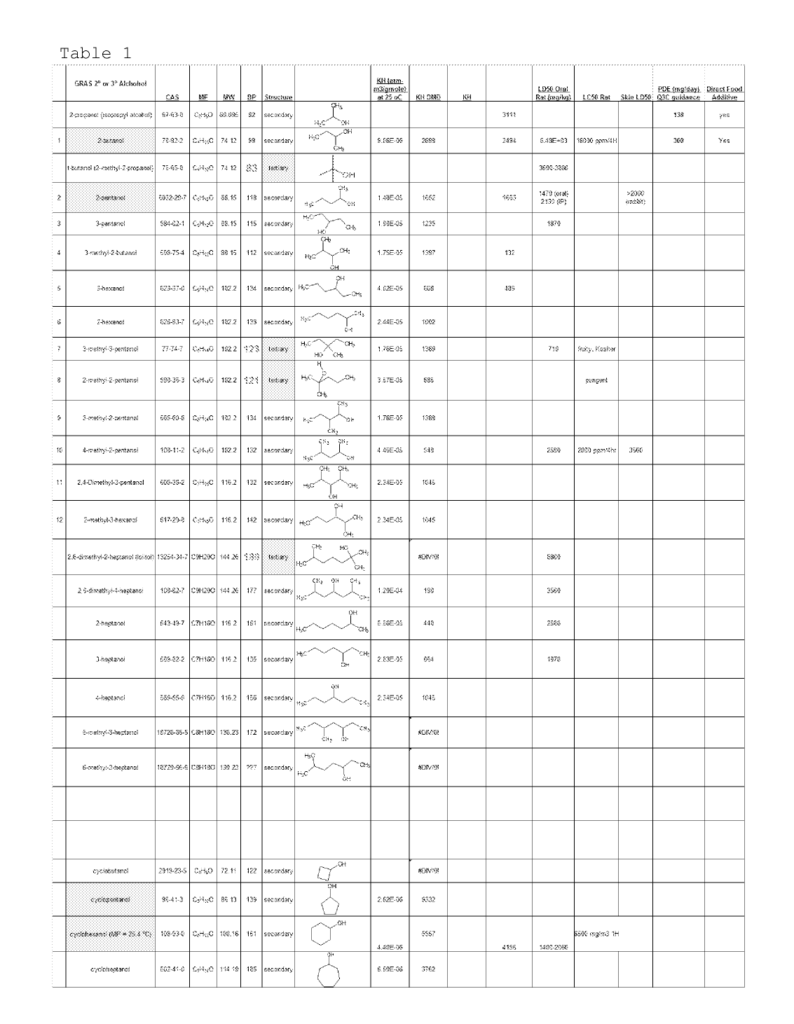

In one embodiment according to this invention, a gel composition used commercially for vaginal or rectal delivery of tenofovir is utilized. This gel comprises 0 (placebo), 0.2, 1, or 5% tenofovir (Gilead Sciences, Inc., Foster City, Calif.) in a gel containing purified water, edentate disodium, citric acid, glycerin, propylparaben, methylparaben, and hydroxycellulose adjusted to pH 4 to 5. (Published Ahead of Print 10 Oct. 2011. 10.1128/AAC.00597-11. Antimicrob. Agents Chemother. 2012, 56(1):103. DOI: Nuttall et al., Pharmacokinetics of Tenofovir following Intravaginal and Intrarectal Administration of Tenofovir Gel to Rhesus Macaques). It will be appreciated by those skilled in the art that different compositions known in the art may be used as the vehicle/substrate for vaginal or rectal delivery of the AEM and API. For example, those skilled in the art are referred to U.S. Pat. Nos. 7,192,607; 7,935,710; 8,367,098 for disclosure on such substrates and procedures known in the art. Hydroxyethylcellulose (HEC), see Example 4 herein and FIG. 4 herein, has been used effectively to deliver an AEM according to this invention for rapid detection of the marker or metabolite thereof in the exhaled breath.

D. AEM and API Delivery Compositions, Methods and Devices for Vaginal and Rectal Delivery

Those skilled in the art will be aware that a wide range of different APIs may be delivered via the rectum or vagina in a wide range of delivery media and mechanisms. Thus, while the terms “microbicide” or “microbicidally active” are generically applied to APIs for delivery by these routes, and while the intent is to include such compounds as tenofovir, emtricitabine, or combinations thereof (e.g. tenofovir disproxil fumarate, marketed by Gilead Sciences under the trade name VIREAD®), emtricitabine, and combinations of emtricitabine and tenofovir, e.g. TRUVADA®), the term is also intended to include any known or hereafter discovered reverse transcriptase inhibitors, protease inhibitors, other mode-of-action antiretroviral APIs and, indeed, any other API for which vaginal or rectal delivery is a known or desired route of medication administration (e.g., valium).

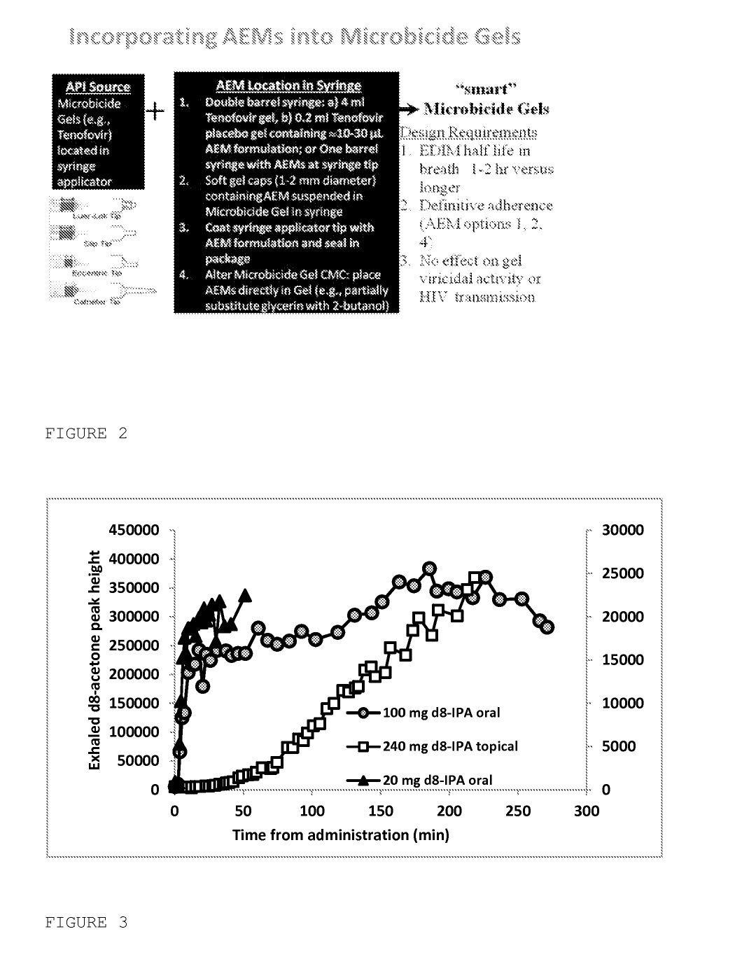

In a preferred embodiment according to this invention, the microbicidal composition according to this invention includes an AEM and the microbicidally active compound is selected from the group consisting of marketed or investigational antiretroviral drugs used either solely or in combination to treat HIV infection, selected from the group consisting of:
A. Nucleoside Reverse Transcriptase Inhibitors (NRTIs) abacavir, abacavir sulfate, azidothymidine, didanosine, dideoxycytidine, dideoxyinosine, emtricitabine, lamivudine, tenofovir disoproxil fumarate, stavudine, zalcitabine, zidovudine;
B. Non-nucleoside Reverse Transcriptase Inhibitors (NNRTIs): delavirdine, efavirenz, etravirine, nevirapine, rilpivirine;
C. Protease Inhibitors (PIs): amprenavir, atazanavir sulfate, darunavir, fosamprenavir calcium, indinavir, lopinavir, nelfinavir mesylate, ritonavir, saquinavir, saquinavir mesylate, tipranavir;
D. Fusion Inhibitors: enfuvirtide;
E. Entry Inhibitors—CCR5 co-receptor antagonist: maraviroc;
F. HIV integrase strand transfer inhibitors: raltegravir; and
G. Combinations thereof.

Where there is any concern about potential negative impact of admixture of an AEM according to this invention with an API for delivery via the rectal or vaginal route, because of stability considerations (e.g. shelf-life, interactions between the API and the AEM and the like), desire to avoid modification of compositions that have already received regulatory approval in the absence of the AEM, or other considerations, the present invention contemplates means for admixture of the AEM at the site of delivery. This is achieved, for example, by maintaining the microbicidally active compound and the AEM in compartments in the drug delivery means such that they are not in contact with each other until delivered vaginally or rectally. Accordingly, in one embodiment according to this aspect of the invention, the API and AEM are maintained, prior to delivery, in separate barrels of a two barreled syringe. Alternate arrangements and embodiments to achieve a similar result include, for example, by including the AEM in (a) a Luer-lock tip which fits over the delivery means, e.g. a syringe, for the API in substrate; (b) in a slip-tip, either coaxially located, eccentrically located, or elongated, as in a catheter tip, which fits over the delivery means, e.g. a syringe, for the API in substrate. Naturally, those skilled in the art will appreciate that in commercial embodiments, such combinations of physical means for keeping the AEM and API separate from each other may be refined and may appear less like syringes than as unitary delivery means, but the operative principles inherent in these non-exclusive examples are the same. In another embodiment according to this aspect of the invention, the AEM is maintained in a softgel capsule which is broken on delivery, e.g. by impact with a plunger, pin or needle tip, or the like, thereby mixing the AEM with vehicle, microbicidally active compound or both, at the site of delivery. Likewise, the intact softgel containing the AEM could be delivered from the syringe along with the microbicidally active compound at the time of product use, and the softgel dissolves in the warm environment of the vagina. In yet another embodiment according to this aspect of the invention, the AEM is coated on a syringe applicator tip which admixes the AEM on delivery of the vehicle and the microbicidally active compound. In yet another embodiment according to this invention, the Chemistry, Manufacturing and Controls (CMC) of a medication is modified to directly accommodate the AEM. For example, for this approach, in the vehicle for a vaginally or rectally administered API, where glycerin is generally a major component of the vehicle, a tiny amount of glycerin is replaced with the AEM, such as 2-butanol. These various delivery options and mechanisms are exemplified in FIG. 2. Yet another means of delivery of the API and AEM may be via a vaginal ring, or similar device. According to this embodiment of this aspect of the invention, a polymeric drug delivery device provides controlled release of drug and AEM for intravaginal delivery over an extended period of time. The drug/AEM delivery device is inserted into the vagina and can provide contraceptive protection, microbicidal protection, and delivery of the AEM. By inclusion of the AEM, and confirming ongoing detection of EDEM in the exhaled breath, clinicians can be assured that the drug delivery device is working correctly and has not been prematurely removed. For rectal delivery, of course, a gel or suppository device/composition is preferred.

With respect to a suppository, the AEM may be admixed with the API and suppository vehicle, or the AEM may be in a separate compartment which is dissolved upon API/suppository delivery, thereby releasing the AEM for detection in the breath or for metabolism to generate the EDEM.

Those skilled in the art will appreciate that while this disclosure focuses primarily on vaginal and rectal delivery of microbicidally effective APIs and confirmation of such delivery, first, other APIs, including but not limited to high value pharmaceuticals, such as small molecules, peptides, proteins, DNA/RNA-based therapeutic agents such as aptamers, as the API. The API delivered according to this invention may have other modes of action, aside from microbicidal efficacy, with adherence being confirmed according to the principles described herein. Second, those skilled in the art will appreciate that vaginal and rectal delivery is but a special case of transdermal delivery, and the principles, methods, compositions, devices and systems according to this invention are relevant to NODFs generally. Finally, as shown in the examples herein below, transdermal (including vaginal and rectal) delivery of APIs may be detected and confirmed in the exhaled breath with exquisite sensitivity when non-ordinary (but preferably non-radioactive) isotopes of certain elements (e.g. hydrogen (i.e. deuterium), carbon, oxygen, nitrogen, sulfur and the like) are included in the AEM. For purposes of the present invention, it should be understood that the preferred non-ordinary isotope is a non-radioactive form, which is distinct from the most abundant isotopic form of a particular element. In this way, not only can background levels of a contaminant in the exhaled breath be distinguished from the actual marker produced by the AEM, but limits of detection in the low parts per billion, down to as low as several parts per trillion are enabled. Use of an infra-red (IR) detector, with or without including mGC or mass-spectrometer based technology (to facilitate discrimination of molecular species based on mass and/or other separation properties) is preferred in such circumstances.

As can be seen from FIGS. 1-4 included in this patent disclosure, the kinetics of appearance and clearance of the markers in the breath are determined for a given AEM delivered by a topical, vaginal or rectal route, and, depending on the concentration of the marker on the breath at any given time, the subject's adherence or non-adherence in taking a particular medication at a particular time and dosage is determined.

In light of this disclosure and the examples which follow, those skilled in the art will appreciate that this invention comprehends within its scope a topical, vaginal or rectal composition adapted for medication adherence monitoring, which includes (a) at least one Adherence Enabling Marker (AEM) which, when delivered topically, vaginally or rectally produces an Exhaled Drug Emplacement Marker (EDEM) which is the AEM itself or a metabolite thereof detectable in the exhaled breath;

(b) a vehicle for vaginal or rectal delivery of an Active Pharmaceutical Ingredient (API) active compound; and
(c) an API. Such a composition according to this invention preferably includes least one or a combination of the following:
a. the API is a microbicidally active compound;
b. the API is a peptide or a protein;
c. the API is a small organic molecule (molecular weight <900-1200 daltons);
d. the API is a DNA/RNA-based therapeutic such as an aptamer;
e. the AEM is a low molecular non-toxic compound that is, preferably, volatile or semi-volatile;
f. the AEM is a secondary or tertiary alcohol with between three and eight carbon atoms;
g. the AEM is a small organic compound (molecular weight <900-1200 daltons), which also serves as the API;
h. the AEM is a peptide;
i. the AEM is a protein; and
j. the AEM is a DNA or RNA molecule.

In any embodiment of the composition according to this invention, any one or combination of the following may be applicable for a given context: the AEM comprises a non-radioactive but non-ordinary isotope; the API is a microbicidally active compound; the AEM is a secondary or tertiary alcohol or a peptide; the AEM is a secondary or tertiary alcohol selected from the group consisting of 2-butanol and 2-pentanol, or combinations thereof, and a peptide selected from peptide T, DAPTA, mDAPTA; the microbicidally active compound is selected from the group consisting of marketed or investigational antiretroviral drugs used either solely or in combination to treat HIV infection, selected from the group consisting of: A. Nucleoside Reverse Transcriptase Inhibitors (NRTIs); B. Non-nucleoside Reverse Transcriptase Inhibitors (NNRTIs); C. Protease Inhibitors (PIs); D. Fusion Inhibitors; E. Entry Inhibitors—CCR5 co-receptor antagonist; F. HIV integrase strand transfer inhibitors; and G. Combinations thereof.

In a related aspect, this invention comprises a system which includes: (a) a SMART® drug comprising or which generates a marker or markers referred to as the Exhaled Drug Emplacement Marker(s) (EDEMs), that appear(s) in the exhaled breath of humans or other vertebrates, to confirm definitive medication adherence, and 2) a SMART® device, which accurately measures the EDEMs and optionally provides medication reminder functions, and orchestrates critical adherence information flow between relevant stakeholders; wherein the SMART® drug is one which is included in a topical, vaginal or rectal composition adapted for medication adherence monitoring, which includes (a) at least one Adherence Enabling Marker (AEM) which, when delivered topically, vaginally or rectally produces an Exhaled Drug Emplacement Marker (EDEM) which is the AEM itself or a metabolite thereof detectable in the exhaled breath; (b) a vehicle for vaginal or rectal delivery of an Active Pharmaceutical Ingredient (API) active compound; and (c) an API. In such a system according to this invention, the SMART® device accurately measures the EDEMs and optionally provides medication reminder functions, and orchestrates critical adherence information flow between the relevant stakeholders (which may include but is not limited to clinical trial monitors, physicians, care provides, insurance companies, remote data storage facilities). Such a SMART® device is preferably selected from the group consisting of miniaturized Gas Chromatography linked to a Metal Oxide Sensor (mGC-MOS), surface acoustic wave (SAW) sensors, infrared (IR) sensor, ion mobility spectroscopy (IMS) sensors, mass spectroscopy, or combinations thereof.

A further related aspect of this invention is a method for definitive monitoring of medication adherence, wherein the medication is adapted for topical, vaginal or rectal administration. The method includes (A) providing to a subject a medication comprising (i) at least one microbicidally active compound; (ii) an Adherence Enabling Marker (AEM) composition, wherein the AEM composition is selected from the group consisting of at least one of: (a) a low molecular non-toxic volatile compound; (b) a secondary or tertiary alcohol with between three and eight carbon atoms; (c) a peptide; (d) a protein; which AEM when delivered topically, vaginally or rectally produces an Exhaled Drug Emplacement Marker (EDEM) detectable in the exhaled breath; and (iii) a vehicle for topical, vaginal or rectal delivery of a microbicidally active compound; and (B) measuring the exhaled breath of the subject with a SMART® device, which accurately measures the EDEM and optionally provides medication reminder functions, and orchestrates critical adherence information flow between relevant stakeholders. In a preferred embodiment of this method, the AEM is selected from the group consisting of the compounds shown in Table I and combinations thereof, a peptide, and a protein. The AEM may also be the microbicidally active compound, as in, for example, when Peptide T, DAPTA, mDAPTA or an analog thereof is delivered according to the method. Alternatively, the AEM is a secondary or tertiary alcohol, selected, for example, from the group consisting of 2-butanol, and 2-pentanol, and combinations thereof; and the microbicidally active compound is selected from the group consisting of: A. Nucleoside Reverse Transcriptase Inhibitors (NRTIs); B. Non-nucleoside Reverse Transcriptase Inhibitors (NNRTIs); C. Protease Inhibitors (PIs); D. Fusion Inhibitors; E. Entry Inhibitors—CCR5 co-receptor antagonist; F. HIV integrase strand transfer inhibitors; and G. Combinations thereof. In practicing the method according to the invention, the SMART® device accurately measures the EDEMs and optionally provides medication reminder functions, and orchestrates critical adherence information flow between relevant stakeholders, and is selected from the group consisting of miniaturized Gas Chromatography linked to a Metal Oxide Sensor (mGC-MOS), surface acoustic wave (SAW) sensors, infrared (IR) sensor, ion mobility spectroscopy (IMS), and mass spectroscopy sensors. In a preferred embodiment according to the invention, the microbicidally active compound and the AEM are not in contact with each other until delivered topically, vaginally or rectally due to (a) being maintained prior to delivery in separate barrels of a two barreled syringe; (b) the AEM being maintained in a softgel capsule which is broken on delivery or dissolved in the body on delivery topically or to the rectum or vagina, thereby mixing the AEM with the vehicle and the microbicidally active compound; or (c) the AEM being coated on a syringe applicator tip which admixes the AEM on delivery of the vehicle and the microbicidally active compound.

In yet a further related aspect of this invention, there is provided a device for topical, rectal or vaginal delivery of an Active Pharmaceutical Ingredient, API, and an Adherence Enabling Marker, AEM, comprising: (a) a reservoir for the API in a vehicle appropriate for delivery of the API to the topical site, including, but not limited to, the skin, the rectum or vagina of an individual; (b) a reservoir for the AEM. Preferably, the reservoir for the API and the reservoir for the AEM provide a barrier such that the API and the AEM are not in contact with each other until such time the API is delivered to the topical site, the rectum or the vagina, at which time the AEM is concurrently delivered to the topical site, rectum or vagina. In specific embodiments of the medication delivery device according to the invention, there is provided a device consisting of: (a) a two-barreled syringe wherein the API and AEM are maintained, prior to delivery, in separate barrels of the two barreled syringe; (b) a Luer-lock tip containing the AEM which fits over the delivery means containing the API; (c) a slip-tip, either coaxially located, eccentrically located, or elongated, as in a catheter tip, which fits over a delivery means for the API; (d) a softgel capsule containing the AEM which is broken on delivery of the API thereby mixing the AEM with the API at the site of delivery; (e) a coating of the AEM on a syringe applicator tip which admixes the AEM on delivery of the API; (f) a vaginal ring comprising a polymeric drug delivery device which provides controlled release of the API and the AEM for intravaginal delivery over an extended period of time; and (g) a suppository wherein the API and the AEM are admixed or are separated from each other by a barrier which breaks or dissolves upon or shortly after emplacement in the rectum. Based on the present disclosure, those skilled in the art are able to determine appropriate pharmaceutically effective doses for delivery according to this invention along with a marker for confirmation of placement of the API. Likewise, based on the present disclosure, those skilled in the art will be able to determine the amount of marker to include with an API for delivery and efficient detection of the marker or metabolite thereof in the exhaled breath. The mass of marker may be from 0.1 micrograms to 100 mg, and any amount between and including these limits, depending on the marker, the mode of delivery and desired rate and sensitivity for detection in the exhaled breath. Further guidance is available with reference to the non-limiting examples which follow.

### EXAMPLES

Having generally described this invention herein above, the following exemplary support is provided to further enable those skilled in the art to practice this invention to its full scope. This detailed written description and enabling disclosure is not, however, intended to be limiting on the invention. Rather, for an apprehension of the scope of the present invention, those skilled in the art are directed to the appended claims and their equivalents.

### Example 1

### Clinical Studies to Optimize and Validate the SMART® Composition, System, Method and Device According to this Invention

Xhale, Inc. submitted its first 510(k) submission to the FDA on Jan. 25, 2013 for clearance of a portable miniature gas chromatograph (mGC). This device was designated a Class I general purpose laboratory instrument that is capable of analyzing gaseous samples (e.g., human breath) for suitable organic molecules of clinical interest. The device detects a wide variety of volatile organic compounds (VOCs), including but not limited to alcohols, aldehydes, ketones, esters, and ethers in a qualitative manner. The ketone, 2-butanone, was selected as a prototypical VOC for detailed device testing according to Clinical and Laboratory Standards Institute (CLSI) protocols. A desktop gas chromatograph (GC), the Hewlett Packard Gas Chromatograph Model 5890A, was used as the predicate device. The mGC is operated by a trained individual, and can be used in the health care, clinical laboratory, or home settings.

In a second 510(k) submission, Xhale will seek clearance of an mGC-based device for use in a breath-based medication adherence monitoring system, termed the SMART® Adherence System. This SMART® mGC device will be used by laypeople, most frequently in their homes, and will definitively document and report, in real-time, adherence to medications in the clinical trial or disease management settings. The mGC used in the SMART® Adherence System was designed to reliably measure 2-butanone in human breath after ingestion of SMART® drugs which have 2-butanol, a 2° alcohol that is designated by the FDA as a food additive (generally recognized as safe [GRAS]), incorporated into a dosage form containing the active pharmaceutical ingredient (API). The ketone, 2-butanone, termed the exhaled drug ingestion marker (EDIM), rapidly appears in breath after ingestion of the SMART® drug containing 2-butanol, due to its efficient enzymatic oxidation by alcohol dehydrogenase (ADH), primarily via the ααADH isoform.

What was not known prior to the present disclosure was whether 2-butanol or other secondary or tertiary alcohols (see Table I) when used as an AEM incorporated into a composition for vaginal, rectal delivery would be an effective SMART® AEM for medication delivery in a manner that preferably does not alter the manufacturing process of the API and causes minimal-to-no effect on its chemistry, manufacturing and controls (CMC), has no impact on the bioavailability of the API, and does not introduce any extra steps in the clinical trial material (CTM) handling process. The formulation approaches used to incorporate the AEM, e.g. 2-butanol, into the API medication form (e.g., vaginal gel or rectal gel, or other compositions known in the art or which hereafter come to be known) are disclosed herein and are found to be both well tolerated and efficient in production of EDEMs (Exhaled Drug Emplacement Markers) on the breath which confirm adherence in the emplacement of the API dosage(s) in the vagina or rectum, or both.

Clinical Study 1:

Using a crossover design in men (rectal route) and women (rectal and vaginal routes), we have identified optimal AEMs for microbicide applications. Breath marker concentration-time relationships following the administration of the vaginal (20% w/w glycerol) and rectal (5% w/w glycerol) versions of TFV placebo gel containing different AEMs (type and dose) are studied.

Rationale:

We hypothesized that the anatomy and physiology associated with the vaginal and rectal routes of administration, compared to oral delivery, will alter appropriate AEM selection, volatile metabolite emanation, and possibly the concentration-time relationship for metabolite exhalation. We have previously published the feasibility for using 2-butyl acetate (15 and 30 mg) and 2-pentyl butyrate (15 and 30 mg) in gel (TFV placebo) administered by the vaginal route to healthy women to assess microbicide adherence using breath. We observed that the parent ester appeared rapidly in the breath, but the 2° alcohol and ketone appeared in much lower concentrations than expected or observed after oral dosing (metabolic order: ester 2° alcohol >ketone). Specifically, 2-butanone concentrations after vaginal administration were about 10-100 ppb, values considerably less than observed after oral dosing of 2-butanol (about 500-1,000 ppb), even accounting for dose differences. We hypothesize that bypass of hepatic first pass metabolic activity largely accounted for this difference. The major gastric and duodenal veins lead directly into the portal circulation that favors rapid enzymatic conversion of AEMs to metabolites by first pass hepatic metabolism. In contrast, the vaginal venous plexus drainage occurs by pudendal veins leading directly to the inferior vena cava, which thereby avoids the first pass effect. Venous drainage of the rectum differs for this structure's inferior and superior portions. The inferior two-thirds of the rectum drain by the inferior and middle rectal veins and then to the pudendal and internal iliac veins, respectively, that thereafter flow to the inferior vena cava. This blood bypasses first pass metabolism. The superior one-third of the rectum, however, is drained by the superior rectal vein that originates at the inferior mesenteric vein, a major tributary of the portal vein. This blood is subject to first pass metabolism. Therefore, each route's first pass effect is unique: large effect (oral), no effect (vaginal), and some effect (rectal). Additionally, differences in mucosal type (stomach/duodenum and rectum: simple columnar; vagina: non-keratinized stratified squamous) may also cause differences in the rate of AEM absorption. Finally, rectal administration of AEMs may be subject to variable absorption due to possible presence of a fecal mass although we previously did not observe that feeding concurrently with consumption of a AEM affected the appearance of breath metabolites after oral administration of AEMs. For these reasons, understanding the nature and mass of AEM required further delineation to adapt the breath-test system for vaginal, rectal and other non-oral routes of administration.

As noted above, previously tested esters were difficult to dissolve in TFV placebo gel due to their hydrophobic nature and frequently caused mild vaginal burning upon application. We herein report identification of superior AEMs for microbicide applications—simple aliphatic GRAS alcohols, including, but not limited to 2° alcohols: 2-butanol and 2-pentanol. Simple (primary) alcohols (e.g. ethanol) are not only known to be readily absorbed through rectal and vaginal mucosa into the systemic circulation, but, unlike esters, are freely soluble in TFV gel due to its high glycerin content. In addition, simple (primary) alcohols such as ethanol are widely listed in FDA's inactive ingredients guide (IIG) for use in a multitude of products given via various routes of administration. For example, the IIG lists ethanol as being present at a maximum potency of 22.448% in a rectal gel product, which indicates alcohols are safe and well tolerated even at high concentrations when given by routes relevant to microbicide product placement. However, use of primary alcohols as AEMs is less than ideal due to differences in metabolic profiles, and use of too-high concentrations of ethanol via vaginal or rectal delivery is known to perturb the epithelial lining, increasing the risk of disease agent passage.

Even at a dose of 2° alcohol (30 mg in 4 ml of TFV placebo gel), the final concentration of 2° alcohol (0.75%) is 30 fold lower than the ethanol content in this marketed gel. As a result, it was not known if the AEM/EDEM would be detected in the exhaled breath.

Methods: Study Design and Enrollment.

After IRB approval, 48 subjects (24 women and 24 men; age 18 years and older) fed ad lib are enrolled and studied by a clinical trial coordinator. Subjects are excluded due to a known allergy to any component of the AEM formulations or pregnancy (assessed by urine dip stick). Written, informed consent is obtained from all subjects. No food, drink, or smoking is allowed 15 min prior to beginning the study or throughout the duration of the study visit. The timing and type of recent food and drink ingestion and cigarette use is noted along with standard subject demographics and past medical history, medications, and smoking history. Four AEMs, including two 2° alcohols, 1 ester, and an AEM placebo are, tested, namely: 2-butyl acetate, 2-butanol, 2-pentanol, and water. Separate cohorts of subjects are randomly assigned to receive a single AEM (6 men and 6 women per AEM). Subjects receiving an AEM are crossed over to receive three doses (3, 10, and 30 mg) and placebo (0.04 ml water) via one route (rectal) and two routes (rectal and vaginal) in men and women, respectively. Thus, each male and female participant receives a total of 4 interventions (rectal route×3 doses of a specific AEM and placebo) and 8 interventions (rectal and vaginal routes×3 doses of a specific AEM and placebo), respectively. Although the AEMs are randomized to subjects, the doses of AEM are not. In contrast, to minimize or eliminate subject discomfort and maximize subject safety (e.g., higher doses of AEM may cause rectal and/or vaginal discomfort), an ascending dose administration order (3, 10, 30 mg) are used. In the event of significant rectal and/or vaginal burning with an AEM at a particular dose, higher doses of that AEM are not further studied in that subject. Study visits ideally occur on consecutive days at approximately the same time with at least 1 day being allowed to elapse between visits. Since 24 men (4 visits/man) and 24 women (8 visits/woman) subjects are enrolled, a total of 288 adherence assessments (96 for men and 192 for women) are achieved.

AEM Justification and Preparation.

We selected two 2° alcohols (2-butanol and 2-pentanol) and one 2° alcohol-based ester (2-butyl acetate) as AEMs for several reasons. First, 2° alcohols generate corresponding ketone metabolites that are known to appear in breath. In contrast, primary (1°) alcohols are rapidly converted to their corresponding aldehyde via β β-alcohol dehydrogenase (ADH) with subsequent rapid conversion to a corresponding, nonvolatile carboxylic acid by aldehyde dehydrogenase (ALDH). Second, metabolism of 2° alcohols occur via αα-ADH that is not subject to environmental influences (e.g., drugs, diet) compared to the cytochrome p450 system. Third, 2° alcohols are metabolized by isoforms of ADH that are not subject to genetic variability. In contrast, 1° alcohols (e.g., n-butanol) are metabolized by an ββ-ADH isoform that has large genetic variation. Reduction of deviations in the breath concentrations of metabolites due to genomic variation is a favorable attribute. Fourth, 2-butanol and 2-pentanol were specifically selected because we have created preliminary data demonstrating that their ketones appear in breath after oral and vaginal administration of precursor molecules, including 2-butyl acetate. Fifth, the selected AEMs are deemed GRAS, food additives by the FDA with well-known safe toxicological profiles and huge safety margins, particularly at the low doses required when these molecules are employed as AEMs. These types of compounds are used in non-oral marketed products at much higher concentrations, and are present in flavored condoms. Therefore, even with chronic use, the AEMs in TFV gel at the levels required for AEM use will not promote HIV transmission, either by altering the integrity of the vaginal or rectal mucosa or by triggering local immune responses/inflammation.

The following AEMs complying with FDA 21 CFR (172.515) are purchased from Penta (Livingston, N.J.): 2-butanol (CAS number 78-92-2), 2-pentanol (6032-29-7), and 2-butyl acetate (105-46-4). For vaginal and rectal administration, the AEM is mixed into vaginal and rectal TFV placebo gel, respectively, and supplied in syringes. TFV placebo gel is from CONRAD (Arlington, Va.). No active drug (TFV) is present in the gels used in this study, although, of course, for active treatment, active API would be included and adherence demonstrated via the SMART® system. Formulations are prepared by a certified compounding pharmacy (e.g. Westlab Pharmacy, Gainesville, Fla.). Using an automatic pipette equipped with a 100 μL gel-loading tip, a prescribed aliquot of an AEM (3, 10, or 30 mg) is inserted through the narrow end of a syringe barrel. Once the end of the tip is in the approximate center of the TFV placebo (4 mL) gel plug, the AEM is dispensed into the gel. This forms a single bead of AEM within the gel plug. The end of the syringe is then capped, and the syringe is vortexed with narrow end down until the gel plug reforms on the bottom of the syringe. Next, the syringe is inverted (plunger side down) and vortexed until the gel plug settles to the plunger side of the syringe. The above steps are repeated four additional times. The test material is labeled and supplied in a single use, disposable, opaque, coded, 10 ml syringe.

Device Preparation and Data Transmission.

12 mGC units are employed in the study. A given subject is randomly assigned a specific mGC for use during all study visits. Each mGC undergoes a complete calibration check (0, 10, 30, 100, 300, and 1,000 ppb standards in human breath in a gas impermeable bag) at the beginning and end of the study for all AEMs and metabolites, and a single point calibration check (0, 10 ppb standard) prior to first use on any given study day by a single mGC expert. The following relevant analytes are readily identified and measured by the current mGC (Tenax® column) in human breath, namely 2-butanone, 2-pentanone, 2-butanol, and 2-pentanol, at retention times of 100, 205, 78, and 180 sec, respectively. All these analytes cause concentration-dependent increases in mGC responses. Data transmission occurs using a wireless router. After each breath into the mGC, a variety of key time-stamped data is stored locally on the device and automatically uploaded to HIPAA-compliant servers, including but not limited to: raw signal data, breath chromatogram, yes/no adherence assessment generated from peak-detection algorithm, image of subject's face for authentication, and mGC conditions.

Protocol.

After a baseline breath sample is obtained (t=0 min), the subjects receive the AEM via the rectum or vagina, and then provide breath samples for mGC analysis at 2, 5, 10, 20, 30, 45, 60, 90, 120 and 180 min post AEM administration. During the course of the study, the subjects are supervised to verify administration of the test articles. In addition, subjects undergo queries from staff following the study about usability and comfort.

Analysis and Anticipated Results:

Breath marker concentration-time relationships are analyzed using a non-compartmental pharmacokinetic model (WinNonlin version 5.2; Pharsight Corporation, St. Louis, Mo.). Performance metrics of the adherence system based on the receiver operating characteristic (ROC) curve plus sensitivity (Se), specificity (Sp), and accuracy determinations are the study endpoints. Additional analysis follows the guidance proffered by the Clinical and Laboratory Standards Institute (CLSI) EP24-A2, entitled “Assessment of the Diagnostic Accuracy of Laboratory Tests Using Receiver Operating Characteristic Curves.” To assess the effectiveness of the use of the adherence system, ROC curves (plots of Se versus 1-Sp) are used to summarize the diagnostic performance of the adherence system using the mGC automated detection algorithm (software) and second mGC expert. The ROC summary performance metric is used as part of the primary analysis, and the Se and Sp are used as secondary endpoints in the analysis. P<0.05 is considered significant. The usability data is analyzed and reviewed.

Interpretation:

Factors including the rate of release of AEMs from the TFV gel, the permeability of the rectal and vaginal mucous to the AEMs, and the degree of AEM first pass metabolism, are key determinants of the AEM and AEM metabolite concentration-time relationships in human breath. Specifically, they determine the rapidity of breath marker appearance, the balance between metabolite and parent molecules in breath, and their persistence in breath. We anticipate the metabolites to rapidly appear in breath (10 min). At equi-doses of AEMs, it is likely that the ketones (2-butanone and 2-pentanone) appear in breath more quickly and at higher concentrations following rectal than vaginal AEM administration, because, unlike the vagina, the rectal blood supply partially drains into the portal vein (first pass metabolism). Based on previous publications, the sensitivity and specificity following oral ingestion of these AEMs was near unity. We hypothesize that the rectal and vaginal routes will also approach unity. In conclusion, results from this study allow an optimal AEM (e.g., minimal effective dose with an optimal tolerability profile) to be selected for both rectal and vaginal applications in men and women.

Clinical Study 2:

Develop strategies (i.e., multiple-barrel syringe applicators) to effectively incorporate the optimal AEM into the placement of rectal and vaginal TFV gels that requires no change in their manufacturing processes, and preserves a favorable concentration-time profile of the breath marker.

Rationale:

The ability to package AEMs into TFV microbicides without changing the manufacturing processes to create them is a major advantage and is required to rapidly allow inclusion of the SMART system in clinical trials. Issues encompassing monitoring of adherence are secondary to persistent stability of TFV given by oral, vaginal, or rectal routes. Although we are not aware of any interactions between the proposed AEMs and TFV placebo gel, the simplest method to assure TFV gel integrity is to prevent their physical contact. We hypothesize that addition of AEMs will have no effects on the concentration-time profiles of breath markers when used in a double-barreled syringe applicator. Note: encapsulation of TFV by other methods (e.g., vaginal tablets, films) could also be developed if desired by the clinical trial community.

Methods: Study Design and Enrollment.

After IRB approval, 12 subjects (6 women and 6 men; age 18 years and older) fed ad lib are enrolled and studied by a clinical trial coordinator. Syringes that encompass dual barrels (Mixpac™, 5 ml Double Syringe 4:1 mixture, OraTech, Riverton, Utah) are purchased. These syringes mix the contents of side-by-side chambers concurrently throughout the total time of administration. In this syringe, 4 ml of TFV placebo gel without AEM is physically separated in a syringe chamber from 1 ml of TFV placebo gel with an AEM incorporated into gel matrix in a second chamber. Thus, the AEM and TFV are separated for an unlimited period of time until administration. The gels are then mixed by a special tip during delivery when the plunger is pushed. To understand if the use of a double barreled syringe impacts the concentration-time profile of the breath marker (vis-à-vis single barrel syringe), the experiments in Clinical Study 1 are repeated for the optimal AEM at a single dose. In this case, however, the men (rectal route) and women (vaginal) are crossed over to receive the following: 1) AEM in 5 ml of TFV placebo gel from a single barrel syringe, and 2) AEM mixed in 1 mL of TFV placebo gel in one chamber and 4 ml of TFV placebo gel in a second chamber. All other subject characteristics, protocol, and devices are the same as in Example 2. Note: Although Clinical Study 1 utilized 4 ml of TFV placebo gel, 5 ml TFV placebo gel is used in this study because in a real world scenario, it would be undesirable to lower the total dose of TFV being administered rectally or vaginally in the 1% TFV gel. In any case, the TFV placebo gel volumes employed in this Example are adjustable as needed.

Analysis and Anticipated Results:

The time-concentration data for breath markers is plotted for each group (multi-versus single-barrel syringe) and analyzed by two-way ANOVA (factor 1: group; factor 2: time) with Tukey correction for multiple pairwise comparisons. We anticipate that the P value of this ANOVA to be <0.05 overall, <0.05 for time, but >0.05 for group with no group by time interactions.

Interpretation:

If the results are as expected, this is interpreted to mean that the markers appear in breath in a similar manner irrespective of whether a single or double barrel syringe is used, indicating excellent mixing of the AEM into TFV placebo gel by the Mixpac™ syringe. Alternatively, a P value for group <0.05 is interpreted as suggesting that the double-barrel syringe technique is different than single barrel. This finding could demonstrate inferior or superior performance depending on if the breath marker emanation is slower and smaller or faster and larger, respectively.

### Example 2

### Vaginal and/or Rectal Delivery of High Value Pharmaceuticals, (Including but not Limited to Small Molecules, Peptides, Proteins, DNA/RNA-Based APIs), and Adherence Monitoring Using the Xhale SMART® System

In recent years, there has been escalating interest in the possibility of delivering microbicidal proteins, antibodies, peptides, nucleotides, nucleic acids, other macromolecules, and the like via the vaginal or rectal route. A brief review of the relevant literature reveals, for example, the following:
1. Amit Kumar Nayak, “ADVANCES IN THERAPEUTIC PROTEIN PRODUCTION AND DELIVERY”, International Journal of Pharmacy and Pharmaceutical Sciences, Vol. 2, Issue 2 (2010), pp. 1-5, which provides a review of therapeutic proteins and methods for delivery of such proteins via various routes, including via pulmonary, nasal, oral, buccal, transdermal, mucosal, rectal and vaginal routes.
2. Ashok. V, et al., “A Review on Vaginal Route as a System Drug Delivery”, Critical Review in Pharmaceutical Sciences, ISSN 2319-1082, Vol. 1, Issue 1, (2012), earthjounrals.org, pp. 1-19, describing advances and challenges to this route of medication delivery.
3. Dey et al., “Protein-Based HIV-1 Microbicides”. Current HIV Research (2013), 11, 576-594, which provides a review of the state of the art of protein-based microbicides, including by vaginal or rectal delivery, including, e.g. using Lactobacillus spp. The article reports that “One of the most important successes in this endeavor has been achieved with the CAPRISA 004 trial in which a 1% vaginal gel formulation of tenofovir, a nucleotide RT inhibitor, was more than 50% effective in reducing HIV acquisition in women with high gel adherence”. This, of course, begs the question of the need for adequate adherence monitoring technology, a solution provided according to the methods and compositions described in this patent disclosure (i.e. by inclusion in such a formulation an Adherence Enabling Marker (AEM) for detection in the exhaled breath of an EDEM. Use of combination microbicides is recommended, (to prevent the establishment of resistant strains, as has been observed in systemic anti-retroviral therapies), including combinations of non-HIV specific agents (cyclodextrins, detergents, surfactants, polyanionic polymers), agents which preserve or restore the physiological cervicovaginal or rectal environment (acidic pH, H₂O₂-producing Lactobacillus), specific anti-HIV drugs (attachment/fusion/entry blockers, e.g. mDAPTA; reverse transcription, integration, proteolytic processing, particle assembly and release inhibitors, e.g. tenofivir; proteins or peptides that specifically or non-specifically inactivate HIV and/or infected cells or block discrete steps in viral replication).
4. Lakshmi Prasanna et al., “Rectal drug delivery: A promising route for enhancing drug absorption”, Asian J. Res. Pharm. Sci. 2012; Vol. 2: Issue 4, Pg 143-149, provides a review of absorption enhancers (e.g. enamine, salicylates and salicylate derivatives, fatty acids, chelating agents, sulfhydryl depeleters, e.g. diethyl maleate, co-administration of protease inhibitors, cyclodextrins, etc), via the rectal route for protein, peptide and other high value pharmaceutical delivery.
5. Patel and Patel, “Vagina as an application site for drug delivery”, Indian Journal of Novel Drug delivery 4(1), January-March, 2012, 17-23, supports advantages to this route of delivery, including avoidance of hepatic first pass effects, systemic delivery, and potential for rapid absorption.
6. Rohan and Sassi, “Vaginal Drug Delivery Systems for HIV Prevention”, AAPS J. March 2009; 11(1): 78-87, notes that microbicides have become a principal focus for HIV prevention strategies, including for local and systemic delivery. This article reviews drug delivery options, including use of vaginal rings, semisolids-ointments, hydrogels, vaginal films, vaginal staples, nanoparticles for targeted microbicide delivery and the like. The need for platforms to ensure use and compliance, and ultimately product efficacy, is an essential conclusion in this publication.
7. Karpenko et al., “Attenuated Salmonella enteritidis E23 as a vehicle for the rectal delivery of DNA vaccine coding for HIV-1 polyepitope CTL immunogen”, Microbial Biotechnology (2012) 5(2), 241-250, (2011), disclosed successful rectal delivery of DNA vaccine and induction of humoral and T-cell responses against HIV-1.
8. Mann et al., “Mucosal Application of gp140 Encoding DNA Polyplexes to Different Tissues Results in Altered Immunological Outcomes in Mice”, PLOS|One, (June 2013), Vol. 8, Issue 6, e67412, disclosed successful nasal, sublingual and vaginal delivery of DNA-PEI polyplexes to prime immune responses.
9. In WO2013/116503, it was disclosed that nucleic acids are detectable in the exhaled breath. For this work, miRNA recovered from exhaled breath condensate was subjected to reverse transcription-polymerase chain reaction (RT-PCR).

In light of these developments in the field of vaginal and rectal medication delivery, including for high-value pharmaceuticals, including, but not limited to, peptides, proteins, DNA and the like, for HIV treatment and other conditions, prophylaxis, either locally at the site of delivery or for systemic delivery of these and other agents, it is apparent that the present invention provides a critical element for success. According to this invention, in this context, having access to definitive data on when, how often, how consistently, and for how long subjects are utilizing vaginally or rectally administered compounds (or other topically applied routes as disclosed herein above), will make critical contributions to evaluations of safety, efficacy and tolerability of various drug treatments and modes of delivery. Providing a nucleic acid based therapeutic agent or marker with another agent, and detection in the exhaled breath of the therapeutic agent or marker, for example by PCR or RT-PCR, provides a significant method for confirming medication adherence. Without reliable data on hand, such studies necessarily include large amounts of guess-work and/or the need for invasive methods of compliance testing and questioning, all of which is counter-productive when dealing particularly with sensitive diseases and modes of medication delivery.

In one exemplary embodiment according to this aspect of the invention, the protein, peptide or other medication of interest for topical delivery, vaginal delivery or rectal delivery is a compound which when successfully delivered, appears in the exhaled breath. For this purpose, different fractions of the exhaled breath may need to be tested—including, for example, the exhaled breath condensate (EBC).

In one preferred embodiment according to this invention, the therapeutic agent itself (referred to as the Active Pharmaceutical Ingredient, or API) acts as the Exhaled Drug Emplacement Marker (EDEM). In one embodiment, the API contains at least one atom representing the most abundant naturally occurring isotope, termed the monoisotopologue, of the API. Additional non-ordinary isotopes may be included in the same molecule and/or in a marker included with the API. In another embodiment, it is desirable for the API to include an easily detectable marker or markers, e.g. a non-ordinary but non-radioactive isotope (selected from, e.g. hydrogen (e.g. deuterium), carbon, nitrogen, oxygen or other non-radioactive isotopes but which have a very low natural abundance). Appearance of the non-ordinary isotope in the exhaled breath is detected by a breath test including a sensor, e.g. IR or mass spectroscopy, or both, which readily distinguishes between compounds based on light absorption, mass, or both, which include naturally occurring isotopes and the same or similar compounds which include the non-ordinary isotope. The benefits of this approach, in addition to providing definitive medication adherence data when the API or markers included in the API are identified in the exhaled breath, is that this also provides definitive evidence that the API has crossed the dermal, vaginal or rectal barriers and delivery to the systemic system has been successful. This is a non-trivial additional benefit to the present invention, particularly when the API is a protein or peptide, since for such compounds knowing that successful delivery has occurred, in addition to knowing the degree of adherence, is critical to determine.

In an alternate, preferred, similar to the above-described non-ordinary isotope incorporation in an API, is the co-administration of a marker with the API. Use of volatiles, semi-volatiles or the like or compounds which give rise to volatiles, has been described in detail above in this patent disclosure. In addition, by selecting a marker molecule which is as similar as possible to the API, the convenience of not having to modify the API is provided while still providing both delivery and adherence data. This is achieved, for example, by including with for example a protein API a protein or peptide marker which includes in its structure a non-ordinary isotope which is readily detectable in exhaled breath when the API plus marker are co-delivered intra-vaginally, rectally or transdermally. A composition according to this aspect of the invention would include, for example, a protein API, and a known amount of either the API or another protein or peptide which acts as a marker protein or peptide with a readily detectable marker included in the marker protein or peptide. One specific example of such an embodiment includes, but is not limited to, delivery of insulin in the presence of a fraction of insulin containing deuterium, or a fraction of albumin or other innocuous protein or peptide including deuterium, including, for example, where the marker protein or peptide is simply methylated to a known extent, preferably with a labile methyl group which preferably includes a known degree of substitution of deuterium for hydrogen.

As means for achieving efficient systemic delivery via transdermal, vaginal and rectal routes evolve, the technology disclosed herein will become increasingly important for delivery of a wide variety of high-value pharmaceuticals, including, but not limited to, delivery of growth hormones, immunomodulators, antibodies, antibody fragments (e.g. Fab), insulins, erythropoietin, factor VIII, vaccines (e.g. hepatitis-B vaccine), interferons, streptokinase, interleukins, protein C, hirudin, GMCSF, somatotropin, endorphins, enkephalins, epidermal growth factor, antitrypsins, aprotinin, lactoferrin, ACE and/or ACE inhibitors, tricsanthin, cerebrosides, and the like, not to mention a host of small molecules for which these routes of delivery are beneficial, carried in gels, suppositories, bioadhesives, microparticles, nanoparticles and the like, with or without permeation enhancers (where such are found to not increase susceptibility to, e.g. infection agent penetration/infection.

In a specific embodiment, this example provides a formulation comprising an adherence marker according to this invention, peptide T, or a derivative thereof, preferably monomeric DAPTA (or an analog thereof, as disclosed and treated in U.S. Pat. No. 8,178,497, herein incorporated by reference, to maintain physiological activity and therapeutic potency), which is D-Ala1-peptide T-amide, in a composition for topical, vaginal or rectal administration. The composition may contain other APIs or just the peptide T, DAPTA, or analog thereof. The peptide T, DAPTA or analog thereof may itself be modified to include a non-ordinary isotope for facile detection in the exhaled breath. The peptide T, DAPTA or analog thereof may be used as a marker for another API co-delivered by this route to a subject in need thereof. Thus, for example, in a highly preferred embodiment, due to its lack of toxicity and efficacy down to nanomolar or lower concentrations (10⁻⁹ to about 10⁻¹⁷ M, see U.S. Pat. No. 8,178,497, column 6, lines 18-27), peptide T, DAPTA, or an analog thereof (e.g. see Pert et al., “RAP-103, a Short Modified Peptide Analog of Monomeric DAPTA, Reduces Pain in a Rodent Model of Peripheral Neuropathy”, (http://www.rapidpharma.com/uploads/media/2009-01_Abstract.pdf), is a potentially ideal candidate as a peptidyl Adherence Enabling Marker for co-delivery of other APIs, including but not limited to microbicidal compounds as disclosed herein. It is anticipated that the peptide T, DAPTA or analog thereof appears in the exhaled breath following successful transdermal, vaginal or rectal delivery of a composition comprising the peptide T, DAPTA or analog thereof, with or without another API, such as a microbicide or drug of choice for achieving HAART (Highly Active Anti-Retroviral Therapy). In this embodiment, the peptide T, DAPTA or analog thereof may be the API and the AEM, it may include or a fraction of the AEM may include a non-ordinary isotope, or it may be present primarily as a marker for another compound.

Following delivery of this composition, the exhaled breath of the subject is monitored using a SMART® device comprising a sensor adapted for detection of the EDEM in the exhaled breath. A fluidic or microfluidic collection module is optionally included to recover exhaled breath condensate, for detection of non-volatile components of the AEM present in the exhaled breath. Alternatively, a volatile, semi-volatile or incipiently volatile AEM, as disclosed herein, is combined with peptide T, DAPTA or an analog thereof for transdermal, rectal or vaginal delivery, with or without another API or microbicidally active compound, and the volatile marker (EDEM) is measured in the volatile component of the exhaled breath. A detector comprising a mini-GC (mGC) plus a MOS sensor is utilized in the latter scenario. Where a non-ordinary isotope is included in the AEM, preferably an IR sensor is used, with or without the need for a mGC separation module or mass spectroscopy. A variant of transdermal delivery for use in accordance with the present invention of peptide T, monomeric DAPTA (mDAPTA), or an analog thereof, is intranasal delivery. For such an embodiment, again, detection in the exhaled breath of the marker according to this invention (whether the peptide T, mDAPTA or analog thereof is the API itself or is being used as the marker/AEM for another API), provides a convenient method for confirming medication adherence.

### Example 3

### Breath Kinetics of Exhaled d6-Acetone and d7-Isopropanol Following the Topical Application of d8-Isopropanol in a Carbomer Gel

Transdermal:

240 mg of d8-isopropanol was mixed with 3 mL of a carbomer-based aloe gel. This gel was applied to an approximately 20 cm² area of the inner left forearm and covered with a Tagaderm occlusive dressing. To further reduce the permeability of the dressing, the transparent section was covered with a small section of teldar polymer prior to use.

Oral:

Either 100 mg d8-isopropanol or 20 mg d8 isopropanol was delivered orally. For oral dosing, 100 or 20 mg of neat d8-isopropanol were placed in a size 4 licap and the licap was swallowed along with 60-100 mL of water. Following administration of d8-isopropanol, exhaled breath was monitored in real time for the presence of d6-acetone and d7-isopropanol using the Orbitrap LCMS.

Results:

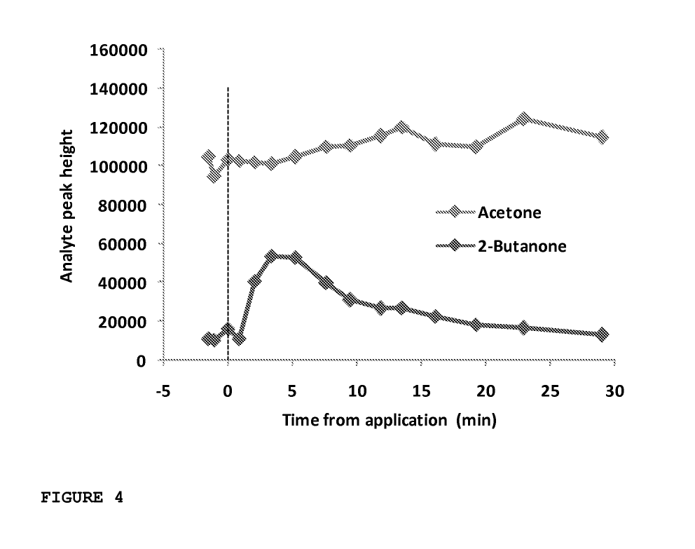

Following application or ingestion, d6-acetone and d7-isopropanol levels were monitored in exhaled breath samples using the LTQ-LCMS. Single full breath samples were administered directly into the modified ESI source at 5 min intervals for ˜4 hours. The ESI source was operated in positive ion mode. A 0.2% NH₄OH:water mobile phase was introduced into the source at a flow rate of 0.1 mL/min during sampling to produce ammonium adducts of the analytes of interest. As can be seen in FIG. 3, by 15 minutes post-ingestion of either 100 mg d8-IPA (left hand axis) or 20 mg d8-IPA (right hand axis), D6-acetone levels in the exhaled breath began to level out and remain at maximum levels for several hours. By contrast, d-8 isopropanol delivered transdermally (right hand axis) resulted in much slower kinetics of appearance of d-6 acetone in the exhaled breath, with a maximum concentration still not achieved by 200 minutes post application.

These data demonstrate that deuterated secondary alcohol, when administered either topically or orally, results in readily detectable deuterated VOCs (d6-acetone) in the exhaled breath for definitive confirmation of medication adherence, albeit with different kinetics of appearance depending on the mode of delivery (oral or transdermal).

### Example 4

### Breath Kinetics of Exhaled 2-Butanone Following the Rectal Application of 2-Butanol in Hydroxyethyl Cellulose (HEC) Gel

Instrumentation and Methods:

40 mg of 2-butanol was mixed with 3 mL of hydroxyethyl cellulose gel in a 3 mL disposable syringe. The entire contents of the syringe were applied rectally as a bolus. Breath acetone and 2-butanone levels were monitored throughout the experiment by delivering single full breath samples directly into the modified ESI source. Two baseline breath samples were taken immediately prior to application and then at 1-3 min intervals thereafter. The ESI source was operated in positive ion mode. A 0.2% NH₄OH:water mobile phase was introduced into the source at a flow rate of 0.1 mL/min during sampling to produce ammonium adducts of the analytes of interest.

See FIG. 4.

Conclusion:

Rectal administration of 2-butanol, even when dissolved in a carrier such as HEC, causes the rapid (2-3 min) appearance of 2-butanone in human breath.

What is claimed is:

1. A topical, vaginal or rectal composition adapted for medication adherence monitoring, comprising:
(a) at least one Adherence Enabling Marker (AEM) which, when delivered topically, vaginally or rectally produces an Exhaled Drug Emplacement Marker (EDEM) which is the AEM itself or a metabolite thereof detectable in the exhaled breath;
(b) a vehicle for topical, vaginal or rectal delivery of an Active Pharmaceutical Ingredient (API) active compound; and
(c) an API.

2. The composition according to claim 1 wherein at least one or a combination of the following apply:
a. the API is a microbicidally active compound;
b. the API is a peptide or a protein;
c. the API is a small organic molecule (molecular weight <900-1200 daltons);
d. the API is a DNA/RNA-based therapeutic such as an aptamer;
e. the AEM is a low molecular non-toxic compound that is, preferably, volatile or semi-volatile;
f. the AEM is a secondary or tertiary alcohol with between three and eight carbon atoms;
g. the AEM is a small organic compound (molecular weight <900-1200 daltons), which also serves as the API;
h. the AEM is a peptide;
i. the AEM is a protein; and
j. the AEM is a DNA or RNA molecule.

3. The composition according to claim 2 wherein said AEM comprises a non-radioactive but non-ordinary isotope.

4. The composition according to claim 2 wherein said API is a microbicidally active compound and said AEM is a secondary or tertiary alcohol or a peptide.

5. The microbicidal composition according to claim 4 wherein said secondary or tertiary alcohol is selected from the group consisting of 2-butanol and 2-pentanol, or combinations thereof, and a peptide selected from peptide T, DAPTA, mDAPTA.

6. The microbicidal composition according to claim 5 wherein said microbicidally active compound is selected from the group consisting of marketed or investigational antiretroviral drugs used either solely or in combination to treat HIV infection, selected from the group consisting of: A. Nucleoside Reverse Transcriptase Inhibitors (NRTIs); B. Non-nucleoside Reverse Transcriptase Inhibitors (NNRTIs); C. Protease Inhibitors (PIs); D. Fusion Inhibitors; E. Entry Inhibitors—CCR5 co-receptor antagonist; F. HIV integrase strand transfer inhibitors; and G. Combinations thereof.

7. A system for monitoring medication adherence comprising:
(a) a SMART® drug comprising or which generates a marker or markers referred to as the Exhaled Drug Emplacement Marker(s) (EDEMs), that appear(s) in the exhaled breath of humans or other vertebrates, to confirm definitive medication adherence, and 2) a SMART® device, which accurately measures the EDEMs and optionally provides medication reminder functions, and orchestrates critical adherence information flow between relevant stakeholders; wherein said SMART® drug comprises said composition according to claim 1.

8. The system according to claim 7 wherein said SMART® device accurately measures the EDEMs and optionally provides medication reminder functions, and orchestrates critical adherence information flow between the relevant stakeholders and is selected from the group consisting of miniaturized Gas Chromatography linked to a Metal Oxide Sensor (mGC-MOS), surface acoustic wave (SAW) sensors, infrared (IR) sensor, mass spectroscopy sensors, and ion mobility spectroscopy (IMS) sensors and combinations thereof.

9. A method for definitive monitoring of medication adherence, wherein said medication is adapted for topical, vaginal or rectal administration, comprising:
(A) providing to a subject a medication comprising
(i) at least one API which is microbicidally active compound, an API selected from a peptide and a protein, an API which is a small organic molecule (molecular weight <900-1200 daltons), an API which is a DNA/RNA-based therapeutic such as an aptamer, and an API which is a small organic molecule (molecular weight <900-1200 daltons);
(ii) an Adherence Enabling Marker (AEM) composition, wherein said AEM composition is selected from the group consisting of at least one of: (a) a low molecular non-toxic compound; (b) a secondary or tertiary alcohol with between three and eight carbon atoms; (c) a peptide; (d) a protein; (e) a DNA or RNA molecule; which AEM when delivered topically, vaginally or rectally produces an Exhaled Drug Emplacement Marker (EDEM) detectable in the exhaled breath; and
(iii) a vehicle for topical, vaginal or rectal delivery of a microbicidally active compound; and
(B) measuring the exhaled breath of the subject with a SMART® device, which accurately measures the EDEM and optionally provides medication reminder functions, and orchestrates critical adherence information flow between relevant stakeholders.

10. The method according to claim 9 wherein said AEM is selected from the group consisting of the compounds shown in Table I and combinations thereof, a peptide, and a protein.

11. The method according to claim 10 wherein said AEM is a secondary or tertiary alcohol is selected from the group consisting of 2-butanol, and 2-pentanol, and combinations thereof; and said microbicidally active compound is selected from the group consisting of: A. Nucleoside Reverse Transcriptase Inhibitors (NRTIs); B. Non-nucleoside Reverse Transcriptase Inhibitors (NNRTIs); C. Protease Inhibitors (PIs); D. Fusion Inhibitors; E. Entry Inhibitors—CCR5 co-receptor antagonist; F. HIV integrase strand transfer inhibitors; and G. Combinations thereof.

12. The method according to claim 9 wherein said SMART® device accurately measures the EDEMs and optionally provides medication reminder functions, and orchestrates critical adherence information flow between relevant stakeholders, and is selected from the group consisting of miniaturized Gas Chromatography linked to a Metal Oxide Sensor (mGC-MOS), surface acoustic wave (SAW) sensors, infrared (IR) sensor, a mass spectrometer, an ion mobility spectroscopy (IMS) sensor, and combinations thereof.

13. The method according to claim 9 wherein said microbicidally active compound and said AEM are not in contact with each other until delivered vaginally or rectally due to (a) being maintained prior to delivery in separate barrels of a two barreled syringe; (b) said AEM being maintained in a softgel capsule which is broken on delivery or dissolved in the body on delivery to the rectum or vagina, thereby mixing said AEM with said vehicle and said microbicidally active compound; or (c) said AEM being coated on a syringe applicator tip which admixes said AEM on delivery of said vehicle and said microbicidally active compound.

14. A device for topical, rectal or vaginal delivery of an Active Pharmaceutical Ingredient, API, and an Adherence Enabling Marker, AEM, comprising:
(a) a reservoir for said API in a vehicle appropriate for delivery of said API to the rectum or vagina of an individual;
(b) a reservoir for said AEM;
wherein said reservoir for said API and said reservoir for said AEM provide a barrier such that said API and said AEM are not in contact with each other until such time that said API is delivered to the rectum or vagina, at which time said AEM is concurrently delivered to the rectum or vagina.

15. The device according to claim 14 selected from the group consisting of: (a) a two-barreled syringe wherein the API and AEM are maintained, prior to delivery, in separate barrels of said two barreled syringe; (b) a Luer-lock tip containing said AEM which fits over the delivery means containing said API; (c) a slip-tip, either coaxially located, eccentrically located, or elongated, as in a catheter tip, which fits over a delivery means for said API; (d) a softgel capsule containing said AEM which is broken on delivery of said API thereby mixing said AEM with said API at the site of delivery; (e) a coating of said AEM on a syringe applicator tip which admixes said AEM on delivery of said API; (f) a vaginal ring comprising a polymeric drug delivery device which provides controlled release of said API and said AEM for intravaginal delivery over an extended period of time; and (g) a suppository wherein said API and said AEM are admixed or are separated from each other by a barrier which breaks or dissolves upon or shortly after emplacement in the rectum.
